(19) United States
(12) Patent Application Publication
(10) Pub. No.: US 2001/0006682 A1
(43) Pub. Date: Jul. 5, 2001

(54) METHOD FOR TREATING PATIENTS WITH SUPPRESSED IMMUNE SYSTEMS

(76) Inventor: SHALOM Z. HIRSCHMAN, RIVERDALE, NY (US)

Correspondence Address: MYRON COHEN, COHEN PONTANI LIEBERMAN & PAVANE, 551 FIFTH AVENUE, SUITE 1210, NEW YORK, NY, 10176

(21) Appl. No.: 08/838,134

(22) Filed: Apr. 15, 1997

(51) Int. Cl. A61K 31/41; A61K 35/16; A61K 35/20; A61K 35/34

(52) U.S. Cl. 424/535; 424/531; 424/548

(57) ABSTRACT

A method of treating patients having suppressed immune systems by perenterally administering Product R, a peptide-nucleic acid preparation, alone or in a combination with one or more immuno-modulators, is disclosed.

### BACKGROUND OF THE INVENTION

I. Field of the Invention

The present invention relates to a method for using Product R as hereinafter defined to treat patients having suppressed immune systems.

II. Description of the Related Art

Product R¹ emerged as an antiviral product in the 1930's. While it was originally believed to be a product composed of peptone, peptides and nucleic acids (fully defined hereafter), the precise composition remains unidentified. Nevertheless, Product R has demonstrated an ability to inhibit rapidly the course of several viral diseases. It is nontoxic, miscible with tissue fluids and blood sera and free from anaphylactogenic properties. 1. The agent is known under the trademark “Reticuloses”, a trademark of Advanced Viral Research Corp.

The immune system is a highly complex system of cells and tissues that requires the cooperation and interaction of a large number of different cell types. The systems of the body comprising the immune system network have been characterized as part of the hematopoietic system, the reticuloendothelial or phagocytic system and the lymphoid system.

Suppression of the immune system is a clinical disorder found in patients having a variety of illnesses. For example, an increase in the incidence of infectious disease has been reported in chronic narcotic users, such as opioid addicts. These addicts appear to have a reduction of T-lymphocyte number and function. In animal models, treatment with opioids has been shown to inhibit lymphocyte proliferative responses, natural killer cell activity, antibody production and circulating levels of interferon. (See Bryant, Henry U. et al., Immunosuppressive Effects of Chronic Morphine Treatment in Mice, Life Sciences, 41:17311 738, 1987).

Cancer patients often exhibit a suppressed immune response. This immunosuppression is most likely caused by agents used to treat the cancer as well as by factors secreted by the cancer cells themselves. Most anti-cancer agents, such a radiation therapy and chemotherapy, inadvertently destroy lymphocytes and other cells important to the immune system. Some tumors, such as the tumors in Hodgkin's disease, release or induce the release of immunosuppressive factors. Hodgkin's disease patients exhibit an abnormally high sensitivity to intracellular parasitic infections such as tuberculosis and herpes virus infections.

Viruses have developed several ways of suppressing the host immune response. Some viruses, such as HIV, target at the host immune system, resulting in a complete destruction of the system. In general, viruses can invade and suppress the host system by infecting T or B cells and abrogate their function, or infecting the thymus and inducing tolerance, or destroying speciallized antigen-presenting cells.

Critically ill patients, such as those with severe burns or complications of sepsis or of multiple trauma, many of whom have been treated with dopamine infusions, often demonstrate suppressed immune function, particularly of cell-mediated immunity. Dopamine is normally administered to patients suffering from hemodynamic imbalances caused by shock syndrome due to disorders such as myocardial infarctions, trauma, endotoxic septicemia, open heart surgery, renal failure and congestive heart failure.

Chronic severe stress has also been associated with depressed immune function. As an example, lymphocytes isolated from individuals exposed to psychosocial stress, such as bereavement, appear to proliferate subnormally, resulting in an impaired immune response. (See Schleifer, S. J., et al., Suppression of lymphocyte stimulation following bereavement, JAMA, 250:374-377 (1983)). Another case of stress-induced immunosuppression can be found in burn patients. The combination of an ineffectual skin barrier to airborne infections in combination with stress-induced immunosuppression could be the cause of a high mortality rate among these patients.

It is believed that the impaired immune response associated with stress is due, in part, to an increased secretion of adrenal corticosteroids from the adrenal glands in response to the stress. However, research has indicated that immunosuppression caused by stress is not solely due to the elevated levels of corticosteroids. A similar suppression of lymphocyte proliferative response in rats following repeated foot shock is evident even after the adrenal glands have been surgically removed.

The mechanism resulting in immunosuppression by corticosteroid hormones is still poorly understood. Direct toxicity to immune cells and their precursors is not seen at the doses of corticosteroids used clinically. While corticosteroids are known to suppress production of immune cell growth factors such as interleukin-2 when added to cultured lymphocytes, this effect only partially accounts for the suppressive effects they have in the whole animal.

Patients who receive exogenous adrenal corticosteroids or synthetic analogs for extended periods of time often develop an impaired ability to secrete endogenous steroid hormones, such as cortisol, due to marked atrophy of the adrenal cortex. When these patients are taken off the exogenous hormone therapy, they can suffer from adrenal insufficiency. Such an insufficiency can be detrimental to patients having an infection, recovering from surgery, or suffering from other stresses because these circumstances give rise to an increased demand for adrenal cortical hormones.

What is needed is a composition that will safely and effectively modulate the immune or adrenal systems of an animal or human to prevent the deleterious effects of stress or corticosteroid-treatment on their function, or to stimulate immune, or bone marrow function when it is impaired due to accidental or therapeutic exposure to radiation or to toxins. Such a composition would preferably be a natural substance to reduce the possibility of adverse side effects.

### SUMMARY OF THE INVENTION

An object of this invention therefore is to provide a method for treating a patient having a suppressed immune system by administering an effective treatment amount of Product R, an antiviral agent composed of peptides and nucleic acids, to the patient.

According to the present invention, the identified patient is treated by administering parenterally an effective treatment amount of Product R from about 5 microliters to about 40 microliters per kilogram of body weight per day in a sterile injectable formulation.

Another object of the present invention is to provide a method for treating a patient having a suppressed immune system by a combination of an effective treatment amount of Product R and one or more immuno-modulators.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### DETAILED DESCRIPTION OF THE PRESENTLY PREFERRED EMBODIMENTS

As used herein, Product R is the product produced according to either of the following methods.

### Method I For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7° C. in a suitable container and gently stir until all the ingredients have been properly wet. Carefully add while stirring about 16.5 g of sodium hydroxide (reagent grade ACS) and continue stirring until sodium hydroxide completely dissolved. Autoclave at about 9 lbs pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/ml, the final volume is approximately 5 liters. The pH is then adjusted with either concentrated HCl (reagent grade ACS) or 1.0 normal NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclave for final sterilization at 240° F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

### Method II For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7° C. in a suitable container and gently stir until all the ingredients have been properly wet. Slowly add while stirring about 11.75 ml of hydrochloric acid (reagent grade ACS) and continue stirring until hydrochloric acid is completely dissolved. Autoclave at about 9 lbs pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/ml, the final volume is approximately 5 liters. The pH is then adjusted with either concentrated HCL (reagent grade ACS) or 35% (w/v) of Noah to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclave for final sterilization at 240 ° F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

For the patient exhibiting immunosuppression, whether caused by viral infections or therapeutic agents or cancer or any other origin, a suitable effective dose of Product R will be in the range of from about 5 microliters to about 40 microliters per kilogram of body weight per day, preferably in the range of about 10 microliters to about 25 microliters per kilogram of body weight per day. Most preferably Product R is administered in an amount of about 30 microliters per kilogram of body weight per day for about one week, followed by about 15 microliters per kilogram of body weight per day in a sterile injectable formulation. The desired dose may be administered as two, three or more sub-doses at appropriate intervals, generally equally spread in time throughout the day. Preferably, the full daily dose is administered in one administration.

Product R may be administered by any suitable injection route including, but not limited to, intravenously, intraperitoneally, subcutaneously, intramuscularly, and intradermally, etc. The presently preferred route of administration is intramuscularly. It will be appreciated that the preferred route may vary with, for example, the condition and age of the recipient.

While it is possible for Product R to be administered as part of a pharmaceutical formulation, it is preferable to present it alone, although it may be administered at about the same time as one or more other pharmaceuticals are independently administered. If Product R is administered as part of a pharmaceutical formulation, the formulations of the present invention comprise at least one administered ingredient, as above defined, together with one or more acceptable carriers thereof and optionally other therapeutic ingredients. The carrier(s) must be “acceptable” in the sense of being compatible with the other ingredients of the formulation and not deleterious to the recipient thereof.

The formulations may conveniently be presented in unit-dose or multi-dose containers, e.g. sealed ampules and vials.

The present invention is also directed to combinations of Product R with one or more immuno-modulators as listed in Table I.

TABLE I
IMMUNO-MODULATORS

| Drug Name | Manufacturer | Indication |
|---|---|---|
| AS-101 | Wyeth-Ayerst Labs. | AIDS |
| | (Philadelphia, PA) | |
| Bropirimine | Upjohn | advanced AIDS |
| | (Kalamazoo, MI) | |
| Acemannan | Carrington Labs, | AIDS, ARC |
| | Inc. (Irving, TX) | |
| CL246,738 | American Cyanamid | AIDS, Kaposi's sarcoma |
| | (Pearl River, NY) | |
| | Lederle Labs | |
| | (Wayne, NJ) | |
| EL10 | Elan Corp, PLC | HIV infection |
| | (Gainesville, GA) | |
| Gamma Interferon | Genentech | ARC, in combination |
| | (S. San Francisco, CA) | w/TNF |
| | | (tumor necrosis factor) |
| Granulocyte | Genetics Institute | AIDS |
| Macrophage Colony | (Cambridge, MA) | |
| Stimulating | Sandoz | |
| Factor | (East Hanover, NJ) | |
| Granulocyte | Hoeschst-Roussel | AIDS |
| Macrophage Colony | (Somerville, NJ) | |
| Stimulating | Immunex | |
| Factor | (Seattle, WA) | |
| Granulocyte | Schering-Plough | AIDS, in combination |
| Macrophage Colony | (Madison, NJ) | w/AZT |
| Stimulating Factor | | |
| HIV Core Particle | Rorer | Seropositive HIV |
| Immunostimulant | (Ft. Washington, PA) | |
| IL-2 | Cetus | AIDS, in combination |
| Interleukin-2 | (Emeryville, CA) | w/AZT |
| IL-2 | Hoffman-La Roche | AIDS, ARC, HIV, in |
| Interleulin-2 | (Nutley, NJ) | combination w/AZT |
| | Immunex | |
| Immune Globulin | Cutter Biological | pediatric AIDS, in |
| Intravenous | (Berkeley, CA) | combination w/AZT |
| (Human) | | |
| IMREG-1 | Imreg | AIDS, Kaposi's |
| | (New Orleans, LA) | sarcoma, ARC, |
| | | PGL |
| IMREG-2 | Imreg | AIDS, Kaposi's |
| | (New Orleans, LA) | sarcoma, ARC, |
| | | PGL |
| Imuthiol Diethyl | Merieux Institute | AIDS, ARC |
| Dithio Carbamate | (Miami, FL) | |
| Alpha-2 | Schering Plough | Kaposi's sarcoma |
| Interferon | (Madison, N,J) | w/AZT: AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| | (Summit, NJ) | |
| Muramyl- | Amgen | AIDS, in combination |
| Tripeptide | (Thousand Oaks, CA) | w/AZT |
| Granulocyte | | |
| Colony | | |
| Stimulating | | |
| Factor | | |
| rCD4 | Genentech | AIDS, ARC |
| Recombinant | (S. San Francisco, CA) | |
| Soluble Human CD4 | | |
| rCD4-IgG | | AIDS, ARC |
| hybrids | | |
| Recombinant | Biogen | AIDS, ARC |
| Soluble Human CD4 | (Cambridge, MA) | |
| Interferon | Hoffman-La Roche | Kaposi's sarcoma |
| Alfa 2a | (Nutley, NJ) | AIDS, ARC, in |
| | combination w/AZT | |
| SK&F106528 | Smith, Kline & | HIV infection |
| Soluble T4 | French Laboratories | |
| | (Philadelphia, PA) | |
| Thymopentin | Immunobiology | HIV infection |
| | Research Institute | |
| | (Annandale, NJ) | |
| Tumor Necrosis | Genentech | ARC, in combination |
| Factor; TNF | (S. San Francisco, CA) | w/gamma Interferon |

It will be understood that the scope of combinations of Product R with immuno-modulators is not limited to the list in the above Table, but includes in principle any combination with any pharmaceutical composition useful for treating patients having immunosuprression.

Preferred combinations are concurrent or alternating treatments of an immuno-modulator and Product R. Product R is preferably administered with the same dosage as it is administered in the absence of one or more immuno-modulators. The dose of immuno-modulators to be co-administered with Product R can be readily determined by those skilled in the art, based on the usual patient symptoms, and severity of the diseases.

Thus, while there have been shown and described and pointed out fundamental novel features of the invention as applied to preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

I claim:

1. A method of treating a patient having suppressed immune system, comprising administering parenterally to said patient an effective treatment amount of Product R in a sterile injectable formulation.

2. The method of claim 1 in which an effective treatment amount of Product R is in a range from about 5 microliters to about 40 microliters per kilogram of body weight per day in a sterile injectable formulation.

3. The method of claim 1 in which an effective treatment amount of Product R is in a range from about 10 microliters to about 25 microliters per kilogram of body weight per day in a sterile injectable formulation.

4. The method of claim 1 in which an effective treatment amount of Product R is about 30 microliters per kilogram of body weight per day in a sterile injectable formulation for about one week, followed by about 15 microliters per kilogram of body weight per day in a sterile injectable formulation.

5. A method of treating a patient having suppressed immune system, comprising co-administering parenterally to said patient an effective treatment amount of Product R in a sterile injectable formulation and one or more immuno-modulators.

6. The method of claim 5 in which an effective treatment amount of Product R is in a range from about 5 microliters to about 40 microliters per kilogram of body weight per day in a sterile injectable formulation.

7. The method of claim 5 in which an effective treatment amount of Product R is in a range from about 10 microliters to about 25 microliters per kilogram of body weight per day in a sterile injectable formulation.

8. The method of claim 5 in which an effective treatment amount of Product R is about 30 microliters per kilogram of body weight per day in a sterile injectable formulation for about one week, followed by about 15 microliters per kilogram of body weight per day in a sterile injectable formulation.
